(19) United States
(12) Patent Application Publication
(10) Pub. No.: US 2011/0306955 A1
(43) Pub. Date: Dec. 15, 2011

(54) MULTIWAVELENGTH LASER APPARATUS FOR SKIN TREATMENT

(75) Inventors: Morten THORHAUGE, Bagsvaerd (DK); Jesper Liltorp Mortensen, Soborg (DK)

(73) Assignee: Advalight Aps, Copenhagen (DK)

(21) Appl. No.: 13/085,085

(22) Filed: Apr. 12, 2011

Related U.S. Application Data
(60) Provisional application No. 61/323,626, filed on Apr. 13, 2010.

(30) Foreign Application Priority Data
Apr. 12, 2010 (DK) PA 2010 00304

(51) Int. Cl. A61B 18/22; A61B 18/20

(52) U.S. Cl. 606/9

(57) ABSTRACT

The invention relates to a laser system for skin treatment and a method for treatment of skin. The laser system comprises a first laser resonator comprising at least a first gain medium for generating a first optical field along a first optical axis. The first laser resonator further comprises a first reflective element and a partly reflective first output coupler. The laser system also comprises a second laser resonator comprising at least a second gain medium for generating a second optical field along a second optical axis. The second laser resonator further comprises a second reflective element and a partly reflective second output coupler. The laser system comprise at least one nonlinear medium for generating a third optical field along a third optical axis by a nonlinear interaction between the first optical field and the second optical field. Finally, the laser system comprises an optical output port capable of delivering the first optical field, the second optical field, and the third optical field to an output, and at least one optical pump source for optically pumping the first gain medium and the second gain medium.

The invention relates to a laser system for use in the treatment of skin and skin conditions.

Due to the increased focus on appearance today one of the central areas for cosmetic treatments is related to so-called rejuvenation. Skin can be affected by a range of biological and ageing effects, and by environmentally induced damages, such as wrinkles, acne, sun damage, reddening, vascular disorders and scarring. Rejuvenation is the combined area of treating these conditions in order to restore the youthful appearance of the skin. One of the most preferred treatments for skin rejuvenation is light based treatments, hereafter referred to as photo rejuvenation.

Several types of light based systems exist today for commercial skin rejuvenation treatments. Light sources intended for these treatments may in general be divided in two groups, consisting of broad wavelength range sources, and narrow wavelength range sources. To the former group belongs systems based on broadband light such as Intense Pulsed Light (IPL) sources, super luminescent light sources and fluorescent light sources. To the latter group belong systems based on laser light sources.

Broadband light sources generally emit light ranging from blue light up into near infrared light; however these may be equipped with appropriate filters to control the spectral content of the emitted light.

Laser light sources utilized for skin rejuvenation include e.g. Nd:YAG lasers emitting at 1064 nm, Alexandrite lasers emitting in the range from about 700 nm to about 750 nm, frequency doubled Nd:YAG lasers emitting at 532 nm, pulsed dye lasers emitting in the range from about 580 nm to about 600 nm, and diode lasers emitting in wavelength bands from about 800 nm to about 900 nm, or from about 900 nm to about 1000 nm or even from about 1300 nm to about 1500 nm.

Various lasers are commonly used in the treatment of a range of skin conditions, such as wrinkles, spot removal, port wine stains, etc. The influence of light on skin tissue may induce micro damages, localized thermal heating or molecular changes. A selection of lasers is typically used, since various wavelengths of laser light have various influences on the skin, e.g. various penetration depths, various absorptions in the layers and components of the skin, etc. Such a setup with multiple separate lasers is bulky and cumbersome to maintain.

Skin rejuvenation as a treatment targets multiple indications, each of which require a characteristic unique wavelength range of the light used for the procedure for maximum efficacy of the treatment.

Treatment of vascular lesions targets hemoglobin in the blood and vascular lesions are thus best treated with light in the wavelength range from about 500 nm to about 600 nm, wherein hemoglobin has a high absorption. Wrinkles and scarring are treated by stimulating collagen growth by triggering a healing response in the skin, which may be caused by inducing non-ablative micro-thermal damages in the skin. To ensure that a sufficient healing response is introduced without ablating effects it is common to use lasers in the range of 900 nm to 1100 nm range, wherein penetration depth is high due to low scattering and tissue absorption. This ensures creation of the desired micro-thermal damages in the lower skin layers required to trigger the healing response.

It may also be desired to target the skin oil secretion in conditions such as acne, where an inhibition of the sebaceous gland (oil gland) activity may lead to an improvement of the condition. Human fat and secreted oil have a significant absorption in the range of 1150-1350 nm and therefore it is of benefit to utilize sources in this wavelength range for selectively targeting sebaceous glands.

Thus to obtain an efficient skin rejuvenation targeting multiple indications it is desirable to use a number of individual wavelength ranges. Typically this may be performed by administering multiple passes with laser sources of different wavelengths. This is however a time consuming procedure which furthermore leads to increased discomfort to the patient simply due to being treated multiple times. Another option is to use broadband light sources such as Intense Pulsed Light (IPL) devices. This is however often not the most efficient either due to the relative large spectral bandwidth of these devices; leading to excessive amounts of energy and thus of heat being deposited in the tissue from spectral ranges not contributing to the skin rejuvenation and giving inferior results in general from having too low energy in the spectral ranges, wherein there is good clinical response, as the total energy is spread across a large spectral range. Adding appropriate filters may remove undesired spectral ranges from the administered light, however this does not address the second problem of low specific fluence in the, often narrow, spectral ranges with therapeutic efficiency.

Furthermore, IPL devices, Nd:YAG lasers and frequency doubled Nd:YAG lasers traditionally have large, uniform spot sizes which for a range of treatments may not be the most optimum. Without being bound by theory it is believed that inducing discrete zones of thermal damage of radius 100 to 1000 or 100 to 1500 microns in diameter in a pattern on the skin with a filling factor not reaching 100 percent may decrease the healing time and thus patient down time. It is therefore desirable to be able to focus the therapeutic energy into small spot sizes.

Thus what is needed is a treatment capable of addressing multiple indications with multiple optimized wavelength ranges in one simultaneous treatment, while retaining the high spectral fluence, and thus high clinical efficacy from using laser based sources, coupled with tight focus on the skin for the decreasing healing time.

Especially for wavelengths in the visible wavelength range, the choice of laser is typically limited to e.g. dye lasers, which require periodic maintenance, including replacement of the dye, which is typically toxic.

Therefore, it is an object of the invention to provide a new laser system for skin treatment that overcomes or ameliorates at least one of the disadvantages of the prior art or which provides a useful alternative thereto.

It is furthermore an object of the invention to provide a method of treatment of skin that overcomes or ameliorates at least one of the disadvantages of the prior art or which provides a useful alternative thereto.

According to a first aspect of the invention, this is obtained by a laser system for skin treatment, the laser system comprising:
a first laser resonator comprising at least a first gain medium for generating a first optical field along a first optical axis, the first laser resonator further comprising a first reflective element and a partly reflective first output coupler,
a second laser resonator comprising at least a second gain medium for generating a second optical field along a second optical axis, the second laser resonator further comprising a second reflective element and a partly reflective second output coupler,
at least one nonlinear medium for generating a third optical field along a third optical axis by a nonlinear interaction between the first optical field and the second optical field,
at least one optical output port capable of delivering one or more of said optical fields to an output, and
at least one optical pump source for optically pumping the first gain medium and the second gain medium. In this way, the laser system is adapted to produce a first optical field having a first wavelength, a second optical field with a second wavelength, and a third optical field with a third wavelength. In the general case, the three wavelengths are different. In a degenerated case, however, two of the wavelengths may be substantially identical. The first and second reflective elements should preferably have a high reflectance at the first and second wavelengths, respectively. Furthermore, each reflective element should be capable of sustaining the optical power level within the corresponding resonator under operation, without damage. The partly reflective output coupler should be capable of reflecting a certain percentage of the power of an incoming field, while allowing the remaining percentage of power to be transmitted through the coupler, disregarding any optical loss.

The first gain medium and the second gain medium should be capable of providing a gain at the first or second wavelength, respectively. Furthermore, the gain media should be capable of sustaining the optical power level within the corresponding resonator under operation. Each laser resonator may comprise further optical components, such as a lens, a mirror, or other passive or active optical components, e.g. for shaping the optical field.

An optical axis is here to be understood as an imaginary line that defines the path along which light propagates through the system. The axis may be deflected by, e.g. a mirror or other optical components.

Wavelength of an optical field is throughout this document to be understood as the vacuum wavelength of the field.

For common general knowledge regarding optics in general and nonlinear optics, see for instance B. E. A. Saleh, M. O. Teich: “Fundamentals of Photonics”, John Wiley & Sons, Inc.; 1991, which is hereby incorporated by reference for all purposes.

In an embodiment of the laser system according the invention, the first laser resonator comprises at least one first Q-switch and/or wherein the second laser resonator comprises at least one second Q-switch, the first Q-switch and/or the second Q-switch being capable of controlling a resonance quality of the first laser resonator and/or second laser resonator, respectively. In this way, light emission from the first and/or second laser resonator may be controlled to provide optical pulses at the first, second, and/or third wavelength.

In a specific embodiment, the laser system comprises a first Q-switch in the first laser resonator and a second Q-switch in the second laser resonator.

In an embodiment of the laser system according the invention, the first Q-switch and/or the second Q-switch is/are of an active type. Thus, a high degree of control over the generated light fields may be obtained by direct control over the Q-switch.

In an embodiment of the laser system according the invention, the active Q-switch comprises an acousto-optical modulator controlled by an external radio-frequency generator, or an electro-optical modulator controlled by a high-voltage generator. In this way, an efficient active Q-switch may be achieved.

In an embodiment of the laser system according the invention, the first Q-switch and/or the second Q-switch are of a passive type. Suitable passive Q-switches could be any type Q-switch comprising a medium with an initial transmittance and a final transmittance, wherein the change in transmittance is essentially caused by the optical intensity of the optical field propagating through the Q-switch. One example of such a passive Q-switch is a saturable absorber.

Naturally, one may also envision a laser system, wherein one or both of the resonators comprise more than one Q-switch, e.g. all being passive, all being active, or some being passive and some being active.

In an embodiment of the laser system according the invention, the passive Q-switch comprises Cr:YAG, V:YAG, and/or Cr:Forsterite. This choice of passive Q-switch material is suitable for first and second wavelengths in the range 1000-1400 nm. Other choices of passive Q-switch materials are readily known by the skilled person, e.g. for use at other wavelengths. See for example: W. Koechner, “Solid-State Laser Engineering”, Sixth Edition, Springer Inc., 2006.

In an embodiment of the laser system according the invention, the first reflective element comprises a mirror and/or the second reflective element comprises a mirror. Thus, an efficient resonator may be achieved, since a wide range of suitable mirrors are available to the skilled person. The mirror may be flat, convex, or concave, depending on e.g. the requirement for beam focusing, to overcome high peak powers, etc.

In an embodiment of the invention, the first reflective element and/or the second reflective element is/are a grating, such as a fiber Bragg grating or a bulk grating.

In an embodiment of the laser system according the invention, the first laser resonator comprises at least one additional first gain medium and/or wherein the second laser resonator comprises at least one additional second gain medium. In this way, advantages of different gain media may be combined within a single laser resonator.

In an embodiment of the laser system according the invention, at least one of the first gain medium and/or the second gain medium comprise(s) a rare-earth doped crystal. Naturally both the first gain medium and the second gain medium may comprise rare-earth doped crystals. Likewise, any additional gain media in the first or second laser resonator may comprise rare-earth doped crystals.

In an alternative embodiment, the first gain medium and/or the second gain medium may be any of the following: a crystal, a gas gain medium, a dye gain medium, a crystal gain medium, a solid phase gain medium, or a semi-conductor laser gain medium.

In an embodiment of the laser system according the invention, the rare-earth doped crystal comprises at least one Nd-doped host material such as Nd:YAG, Nd:YAP, and/or Nd:GdVO₄.

In an alternative embodiment, the gain medium comprises other rare-earth or transition metal dopants, such as Er, Cr, Ho, Yb, Tm. Other host materials may be glass or crystal materials such as KGW, YVO4, YLF, Forsterite, LiCAF, ZBLAN, or other fluoride or silica glasses.

Other choices of gain media and host materials are known to the skilled person, e.g. for use at other wavelengths. See for example: W. Koechner, “Solid-State Laser Engineering”, Sixth Edition, Springer Inc., 2006.

In an embodiment of the laser system according the invention, the first laser resonator is adapted for lasing with a first wavelength in the range from about 1020 nm to about 1080 nm, and wherein the second laser resonator is adapted for lasing with a second wavelength in the range from about 1300 nm to about 1350 nm. Light within these wavelengths ranges is known to be particularly useful in treatments of various skin problems.

In an embodiment of the laser system according the invention, the laser system further comprises an exposure control device. Examples of suitable exposure control devices are a mechanical shutter, an acousto-optical device capable of deflecting the emitted beam onto a beam dump, an electro-optical device capable of altering the polarization state of the beam in combination with a device capable of passing a given polarization state onto the output port of the laser system, while being capable of deflecting the orthogonal polarization state onto a beam dump.

In an embodiment of the laser system according the invention, the laser system further comprises at least one output selector for selecting the first optical field, the second optical field, and/or the third optical field to be delivered to the output port. In this way, the relevant wavelengths for skin treatment may be delivered to the output port, while any unwanted wavelengths may be blocked.

In an embodiment of the laser system according the invention, the output selector comprises a mirror selector and a plurality of selection mirrors, the mirror selector being capable of selectively positioning any of the selection mirrors so that the incident first optical field, second optical field and third optical field are divided into reflected fields and transmitted fields, and wherein at least one of the reflected fields or the transmitted fields is/are delivered to the output port. Thus, by using a plurality of selection mirrors having different reflectance and/or transmittance at the first, second, and third wavelength, the wavelengths delivered to the output port may be controlled. In this way, an output selector is achieved, which is particularly suitable for high optical peak powers.

The skilled person will see that the mirror selector may be driven in a multitude of ways, e.g. manually by a dial, by a stepper motor, by a gear.

For instance, a first selection mirror may have a high reflectance at the first wavelength, but low reflectance at both the second and third wavelength. A second selection mirror may have high reflectance at both the first and second wavelengths, but low reflectance at the third wavelength. A third selection mirror may then have high reflectance for all three wavelengths. Thus, when reflected fields are delivered to the output port, primarily light having the first wavelength is output when the first selection mirror is active, primarily light having the first and second wavelength when the second selection mirror is active and light at all three wavelengths when the third selection mirror is active. Alternatively, when transmitted fields are delivered to the output port, primarily light having the second and third wavelength are delivered when the first selection mirror is active, primarily light having the third wavelength is delivered when the second selection mirror is active, and a reduced output is obtained when the third selection mirror is active. Other combinations of high and low reflectivity and corresponding low and high transmittance for selection mirrors at the three wavelengths are evident for the skilled person.

High reflectivity may here be at least about 50%, such as at least about 75%, or at least about 90%, or even at least about 99%. High transmittance may be at least about 90%, or at least about 95%, or even at least about 99.5%.

In embodiments, wherein the reflected fields are delivered to the output port, the transmitted fields may be delivered to a beam dump, or alternatively to a secondary output port, monitoring port, or the like. Correspondingly, in embodiments, wherein the transmitted fields are delivered to the output port, the reflected fields may be delivered to a beam dump, or alternatively to a secondary output port, monitoring port, or the like.

In an embodiment of the laser system according the invention, the output selector comprises a plurality of filters, wherein each filter allows one or more of the first optical field, the second optical field, or the third optical field to be transmitted through said filter, and wherein the output selector further comprises a filter selector for selecting which filter or filters is/are positioned in front of the output port. In this way, a mechanically simple output selector is achieved, wherein the requirements for alignment are relaxed.

In an alternative embodiment, the output selector may comprise one or more gratings or other devices for spatially separating the first, second, and third optical fields, individually or in combinations.

In an embodiment of the laser system according the invention, the first laser resonator and the second laser resonator are arranged to have substantially overlapping optical axis in a common section over at least a part of a length of the first laser resonator and a part of a length of the second laser resonator, and wherein the first output coupler and the second output coupler are provided as a common output coupler. In this way, a compact system may be achieved, since one or more optical components may be shared between the two resonators.

For instance, the common output coupler may be a mirror having a reflectance at a wavelength of 1064 nm of at least about 60%, such as at least about 65%, at least about 70%, at least about 75%, or even at least about 80%, and having a reflectance at a wavelength of 1319 nm of at least about 85%, or at least about 90%, or even at least about 95%. Such mirrors are readily created by known arts, and may for example be obtained by coating non-absorbing, transparent substrates with multiple layers of dielectric materials such as fluorides and oxides.

In an embodiment of the laser system according the invention, the first Q-switch and the second Q-switch are comprised as a common Q-switch. Thus, a compact and economic system may be achieved, since a single Q-switch may be used. Furthermore, the common Q-switch may be used to synchronize the first laser resonator and the second laser resonator.

In an embodiment of the invention, the first laser resonator and the second laser resonator comprise at least one additional common Q-switch located in the common section, which additional common Q-switch may be of a passive or an active type.

In an embodiment of the invention, the first laser resonator and/or the second laser resonator comprise at least an additional Q-switch on the first and/or second optical axis but away from the common section.

In an embodiment of the laser system according the invention, the nonlinear medium comprises at least one nonlinear crystal. In an alternative embodiment, the nonlinear medium comprises two, three, or more nonlinear crystals. In this way, problems such as back-conversion of the first optical field back into the first optical field and/or second optical field may be mitigated.

In an alternative embodiment, the nonlinear medium comprises a highly nonlinear fiber.

In an embodiment of the laser system according the invention, the nonlinear crystal is chosen from LBO, BBO, KTP, periodically poled (PP) LN, or LT. These choices of nonlinear crystals are suitable for generation of light in the visible range, e.g. yellow light from infrared light.

In an embodiment of the laser system according the invention, the nonlinear medium is capable of generating the third optical field by sum frequency generation from the first optical field and the second optical field. In this way, the third optical field may conveniently be generated from the first and second optical fields. Furthermore, this choice of nonlinear process may alleviate difficulties in obtaining laser light at shorter wavelengths, e.g. within the visible range, by generating this light from light at longer wavelengths, e.g. in the infrared range.

In an embodiment of the laser system according the invention, the laser system comprises at least one first optical pump source for pumping the first gain medium and at least one second optical pump source for pumping the second gain medium. In this way, a simple system may be achieved, wherein control of the optical power in the first optical field and the second optical field may be individually controlled by adjusting the power of the first pump source or the second pump source, respectively.

In an embodiment of the laser system according the invention, the first gain medium is pumped substantially along the first optical axis and/or the second gain medium is pumped substantially along the second optical axis. End-pumping the gain media in this way is generally more pump efficient than pumping, e.g. normal to the optical axis.

In an alternative embodiment of the laser system according the invention, the first gain medium and/or the second gain medium is side-pumped.

In an embodiment of the laser system according the invention, the pump source comprises one or more laser diodes. Such diodes are both inexpensive, mechanically robust, and are practically maintenance free.

In a specific embodiment, the one or more laser diodes of a pump source emit(s) laser light with a wavelength in the range from about 800 nm to about 900 nm, such as from about 805 nm to about 815 nm or from about 880 nm to about 890 nm, or even about 808 nm or about 885 nm.

In an embodiment of the laser system according the invention, the laser system further comprises a hand piece, the hand piece being optically connected to the output by a beam delivery component. In this way, the optical output from the laser resonators and the nonlinear component may conveniently be delivered to a point of treatment on, e.g. the skin of a patient.

In an embodiment of the laser system according the invention, the beam delivery component comprises at least one of: an optical waveguide, an optical fiber, an articulated arm, and/or at least a first delivery mirror. In this way, a particularly user-friendly laser system may be achieved.

In an embodiment of the laser system, the hand piece is adapted to deliver laser light from the output onto a treatment area of skin. The hand piece may be configured for scanning the laser light in a pre-set pattern of individual target skin areas covering said treatment area. Alternatively, the treatment area may be a single target skin area.

In an embodiment of the laser system, the fiber has a core diameter in the range from about 50 μm to about 400 μm.

According to a second aspect of the invention, this is obtained by a method for treatment of skin, the method comprising:
selecting a target skin area to be treated,
positioning a beam delivery device in proximity to the target skin area,
irradiating the target skin area with laser light having at least three distinct wavelengths simultaneously or in succession. By delivering more than one wavelength simultaneously or in rapid succession, an improved treatment may be achieved. The beam delivery device may, e.g. be a hand piece.

In an embodiment of the method of treatment according to the invention, the target skin area is irradiated with laser light having at least three distinct wavelengths simultaneously or in succession.

In an embodiment of the method of treatment according to the invention, the target skin area is irradiated by at least one pulse of the laser light, the laser light having at least three distinct wavelength components suitable for heating constituents in the skin.

In an embodiment of the method of treatment according to the invention, the treatment is cosmetic. For example the method may be used for the purely cosmetic treatment of wrinkles or fine lines in skin, removal of freckles, etc.

In an embodiment of the method of treatment according to the invention, the treatment is photo rejuvenation of skin.

In an embodiment of the method of treatment according to the invention, the distinct wavelengths are chosen to be a first wavelength in the range from about 570 nm to about 610 nm, a second wavelength in the range from about 1020 nm to about 1100 nm, and a third wavelength in the range from about 1140 nm to about 1220 nm or from about 1300 nm to about 1400 nm, respectively.

In one embodiment, the wavelengths being about 598 nm, about 1064 nm and about 1195 nm, respectively.

In another embodiment, the wavelengths being about 589 nm, about 1070 nm and about 1178 nm, respectively.

In an embodiment of the method of treatment according to the invention, the distinct wavelengths are chosen from the ranges from about 580 nm to about 600 nm, from about 1020 nm to about 1080 nm, and from about 1300 nm to about 1350 nm, respectively, such as about 589 nm, about 1064 nm, and about 1319 nm, respectively, or such as about 593 nm, about 1064 nm, and about 1341 nm, respectively, or even such as about 598 nm, about 1079 nm, and about 1341 nm. These wavelengths are commonly used separately in treatment of various skin problems, and have different ways of interacting with the skin layers due to different absorption/penetration of the wavelengths into the layers. For instance, light with wavelengths in the range from about 580 nm to about 600 nm may be used for treatment of minor vessels, red discoloration of skin, hyper-pigmentation, and to stimulate collagen growth. Light with wavelengths of about 1064 nm or about 1079 nm has a very good penetration due to the low absorption in melanin, hemoglobin and water and may be used to stimulate collagen growth and treat deeper lying vessels. Light with wavelengths of about 1319 nm or 1341 nm has a good penetration, but higher absorption in water and fatty tissue than, e.g. 1064 nm or 1079 nm light, and may be used to improve skin elasticity and to stimulate collagen growth.

In an embodiment of the method of treatment according to the invention, the laser light is provided by a laser system according to any of the abovementioned embodiments of the invention. In this way, an improved treatment is obtained, while advantages such as compactness, ease of use, and/or economic benefits of the laser system are maintained.

An embodiment of the method of treatment according to the invention further comprises delivering said three distinct wavelength components simultaneously.

An embodiment of the method of treatment according to the invention further comprises irradiating the target skin area for a duration in a range from about 5 ms to about 300 ms, such as from about 10 ms to about 200 ms, or even from about 20 ms to about 100 ms, or from about 10 ms to about 40 ms.

In an embodiment of the method of treatment according to the invention, the laser light delivers to the target skin area a total radiant exposure in a range of about 15 J/cm² to about 150 J/cm² for each distinct wavelength.

In an embodiment of the method of treatment according to the invention, the laser light delivers to the target skin area a radiant exposure in a range from about 5 J/cm² to about 100 J/cm² per distinct wavelength. For example such that the energies are in the range from about 5 J/cm² to about 50 J/cm² for each distinct wavelength.

In an embodiment of the method of treatment according to the invention, the laser light delivers to the target skin area a radiant exposure in a range from about 10 J/cm² to about 25 J/cm² for said first wavelength, from about 20 J/cm² to 50 J/cm² for said second wavelength, and from about 10 J/cm² to about 25 J/cm² for said third wavelength. For example such that the energy is in the range from about 15 J/cm² to about 25 J/cm² for the distinct wavelength of 589 nm, and from about 30 to about 50 J/cm² for the distinct wavelength of 1064 nm, and 15-25 J/cm² for the distinct wavelength of 1319 nm. Or, for example such that the energy is in the range from about 15 J/cm² to about 25 J/cm² for the distinct wavelength of 593 nm, and from about 30 J/cm² to about 50 J/cm² for the distinct wavelength of 1064 nm, and from about 15 J/cm² to about 25 J/cm² for the distinct wavelength of 1341 nm.

In an embodiment of the method of treatment according to the invention, the pulse delivers to the target skin area a radiant exposure in a range from about 10 J/cm² to about 25 J/cm² for said first wavelength, from about 20 J/cm² to about 50 J/cm² for said second wavelength, and from about 20 J/cm² to about 50 J/cm² for said third wavelength. For example such that the energy is in the range from about 15 J/cm² to about 25 J/cm² for the distinct wavelength of 589 nm, and from about 30 J/cm² to about 50 J/cm² for the distinct wavelength of 1050 nm, and from about 30 J/cm² to about 50 J/cm² for the distinct wavelength of 1178 nm.

In an embodiment of the method of treatment according to the invention, the distinct wavelengths are delivered in a continuous wave (CW) or quasi-CW mode, wherein the laser beams are emitted continuously. The light beam onto the skin may e.g. be regulated in duration by means of a mechanical shutter device.

In an embodiment of the method of treatment according to the invention, the light at least at one of the distinct wavelengths comprises a pulsed laser beam forming a pulse train having a pulse repetition frequency.

In an embodiment of the method of treatment according to the invention, the pulse repetition frequency is in the range from about 8 kHz to about 25 kHz.

In an embodiment of the method of treatment according to the invention, the pulse repetition frequency is in the range from about 25 kHz to about 75 kHz.

In an embodiment of the method of treatment according to the invention, the laser light is delivered to a treatment area comprising multiple target skin areas by a hand piece. The hand piece receives a beam from a light source such as to deliver the beam onto the treatment area. The method further comprises that the hand piece scans the beam in a pre-set pattern of individual target skin areas covering the treatment area. The pattern may be pre-set by the hand piece manufacturer. Alternatively, the pattern may be selectable among a number of pre-programmed patterns, or even be directly programmable, e.g. by a user of the laser system.

In an embodiment of the method of treatment according to the invention, the scanning pattern is a rectangular pattern of individual target skin areas “spots”, with a pattern consisting of 3 to 15 spots in each direction, such as for example a 5×5 pattern, or for example a 6×6 pattern, or for example a 7×7 pattern, or for example a pattern with variable spot density in the range of 3-to-15 by 3-to-15 spots.

In an embodiment of the method of treatment according to the invention, the individual target skin areas of said pre-set scanning pattern are addressed in a non-sequential order.

In an embodiment of the method of treatment according to the invention, the non-sequential order includes not irradiating neighboring target skin area positions in direct sequence. For example, the target skin areas in a scanned pattern may be accessed in a pre-programmed pseudo-random order in such a way as to avoid two neighboring target skin areas being irradiated in a direct sequence immediately after each other. Avoiding exposure on two neighboring target skin areas in sequence reduces risk for general thermal damages to the treatment area.

In an embodiment of the method of treatment according to the invention, a dwell time at said individual target skin area positions is in the range from about 5 ms to about 100 ms.

In an embodiment of the method of treatment according to the invention, the scanned treatment area substantially forms a rectangle with an extent along each side in the range from about 3 mm to about 10 mm, such as for example a 3 mm-by-3 mm area, or for example a 5 mm-by-5 mm area, or for example a 5 mm-by-10 mm area, or even for example a 10 mm-by-10 mm area.

In an embodiment of the method of treatment according to the invention, a size, such as a diameter or a greatest extent of the target skin area is selected to be in the range from about 0.8 mm to about 5.0 mm, such as about 1.0 mm, such as about 2.0 mm, such as about 3.0 mm, or even such as about 4.0 mm. The size of the target skin area being the spot size on the skin of the laser light, i.e. the irradiated spot on the skin.

In an embodiment of the method of treatment according to the invention, the diameter or greatest extent of the individual target skin areas on the skin is in the range from about 0.4 mm to about 1.2 mm. The size of the target skin area being the spot size on the skin of the laser light, i.e. the irradiated spot on the skin.

In an embodiment of the method of treatment according to the invention, the diameter or greatest extent of the individual target skin areas on the skin is in the range from about 0.05 mm to about 0.4 mm. The size of the target skin area being the spot size on the skin of the laser light, i.e. the irradiated spot on the skin.

The invention will be explained more fully below in connection with a preferred embodiment and with reference to the drawings in which:

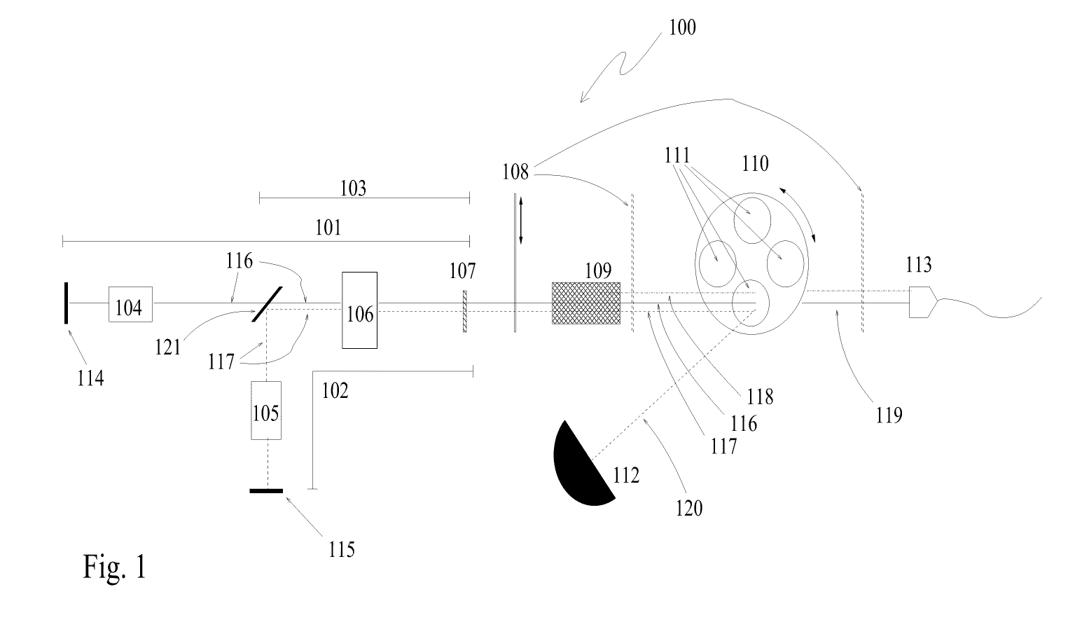

FIG. 1 shows an embodiment of a laser system according to the invention;

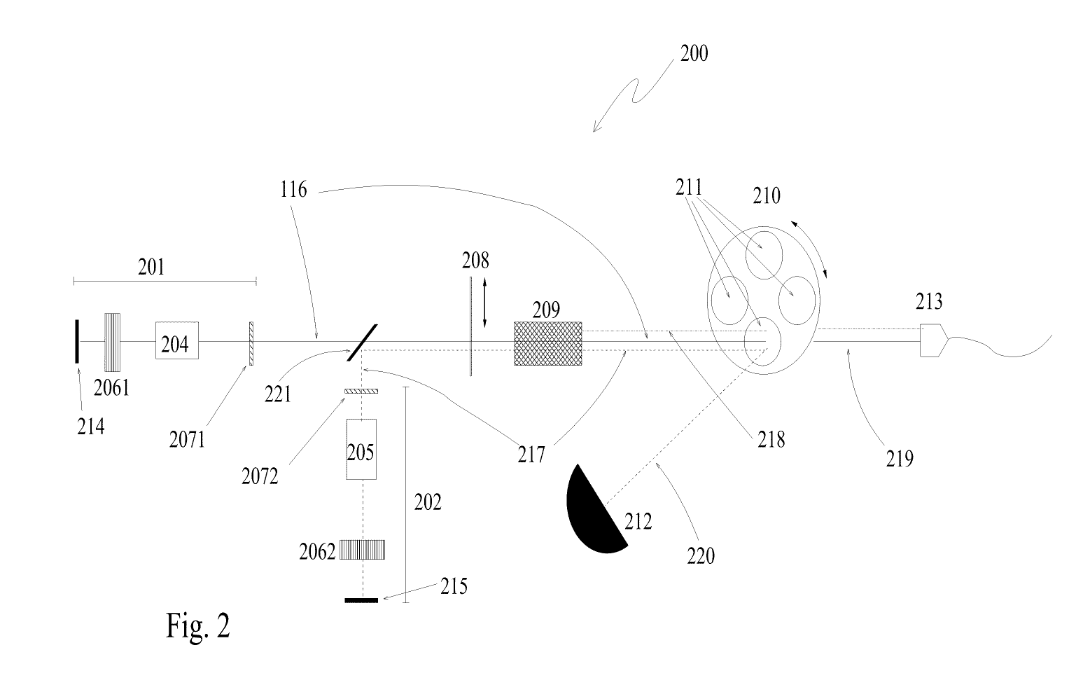

FIG. 2 shows another embodiment of a laser system according to the invention;

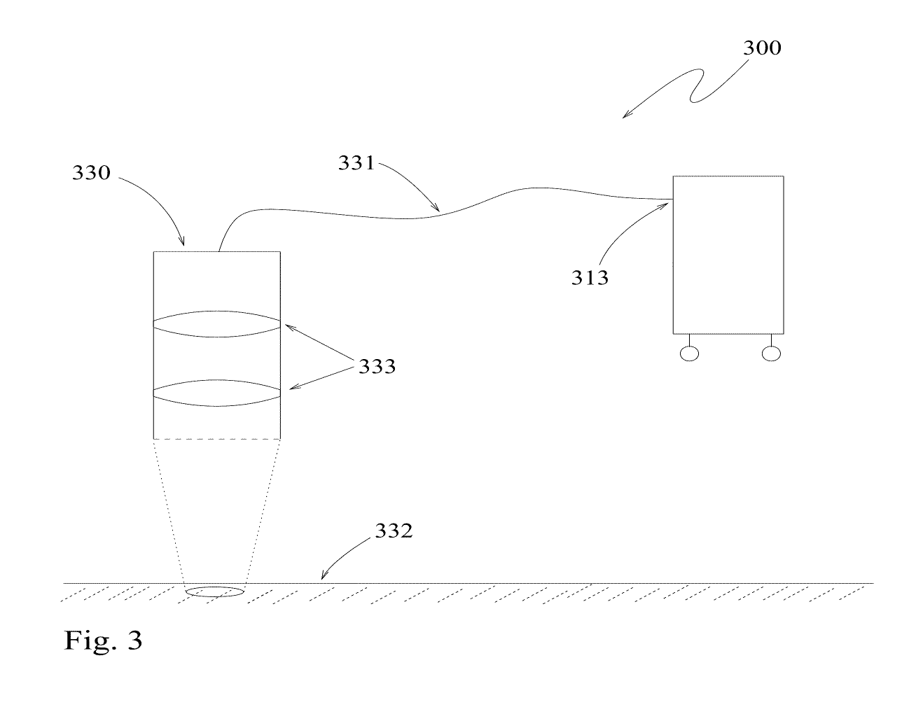

FIG. 3 shows yet another embodiment of a laser system according to the invention;

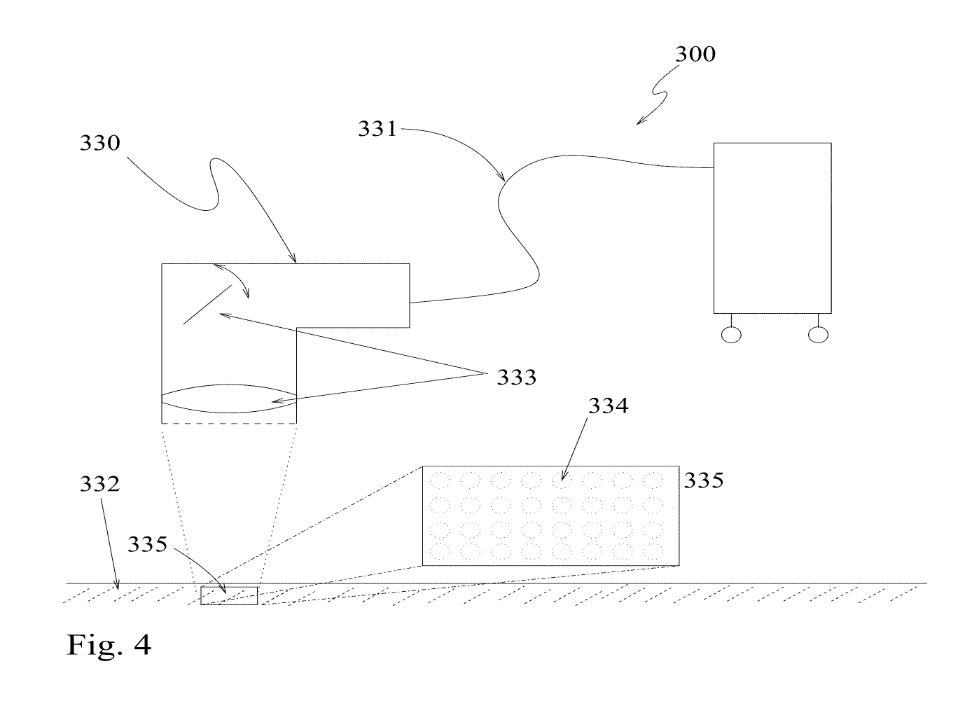

FIG. 4 shows another embodiment of a laser system according to the invention;

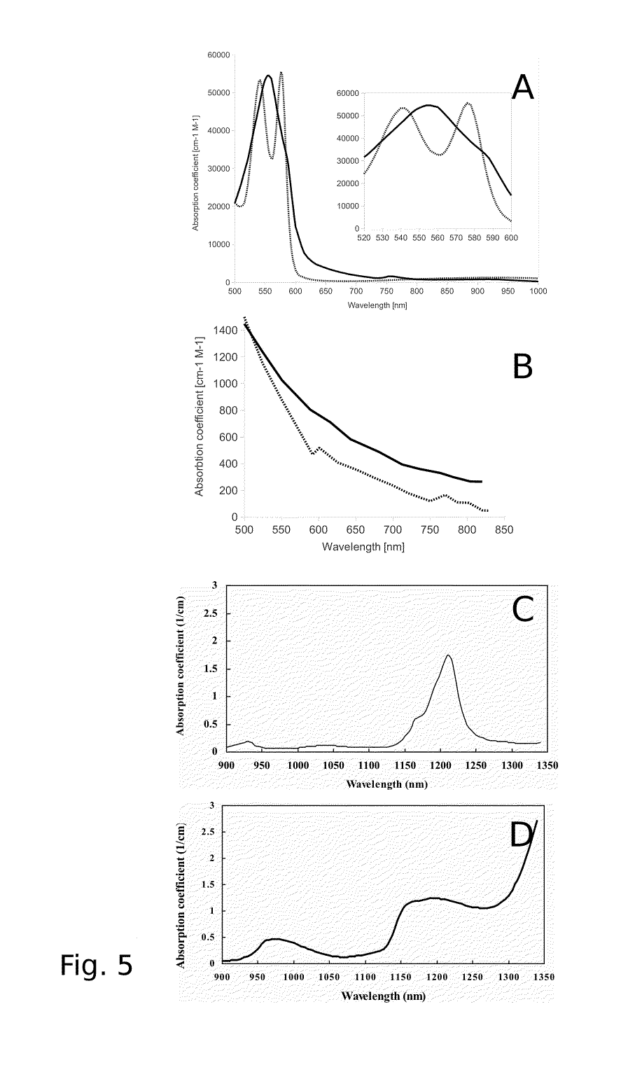

FIG. 5 shows absorption coefficients of different constituents of skin;

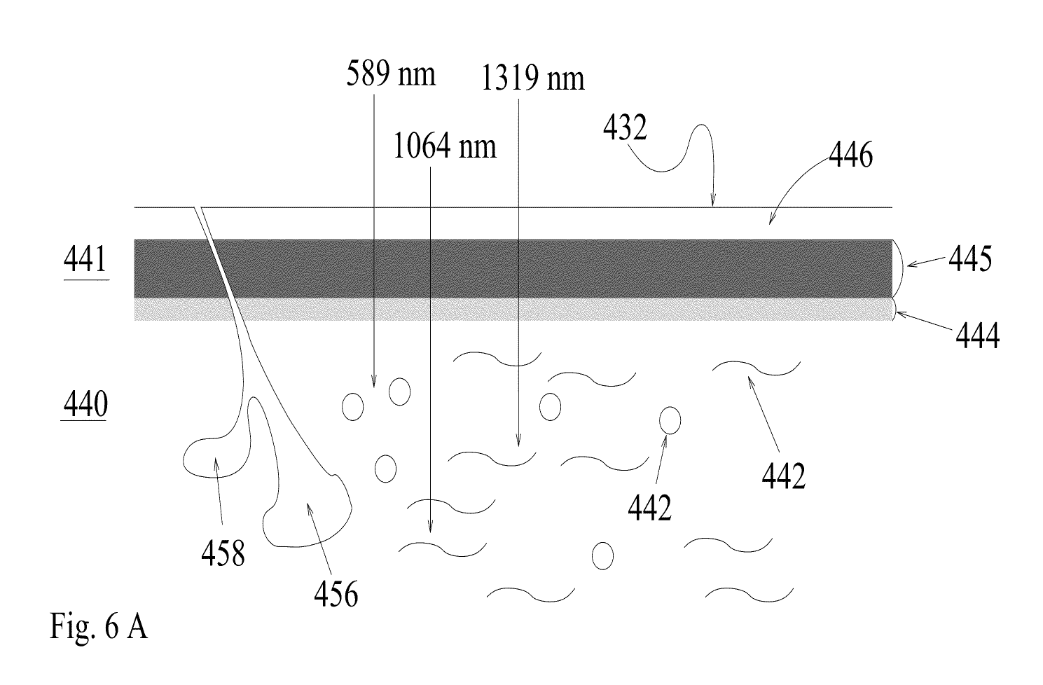

FIG. 6 shows schematic cross sections of skin; and

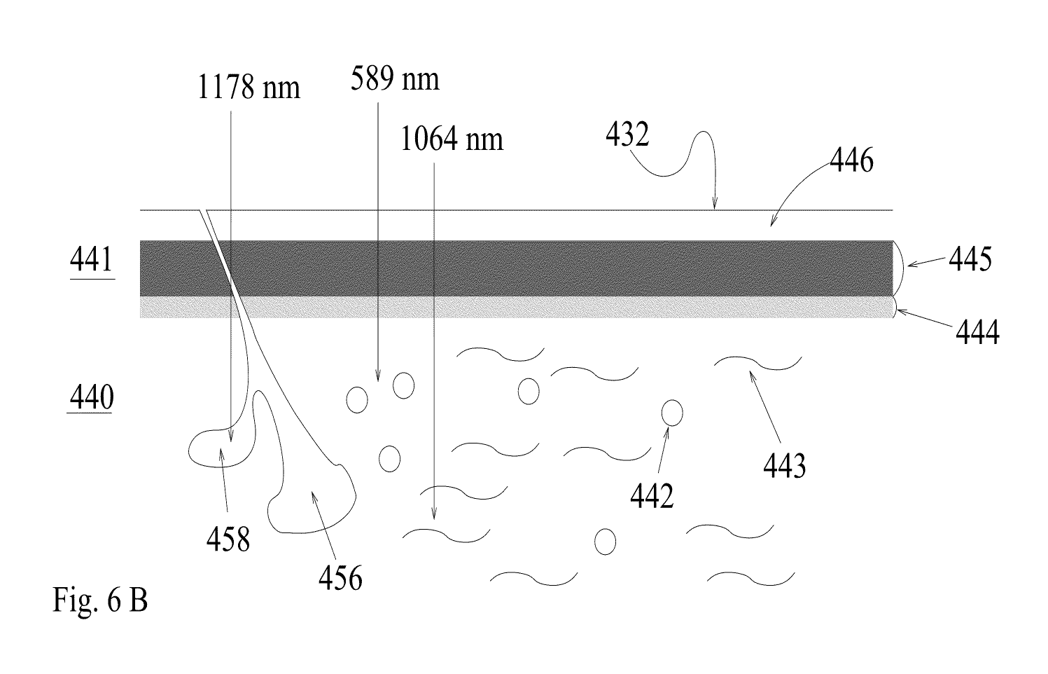

FIG. 7 shows possible output combinations from the inventive system.

The figures are schematic and may be simplified for clarity. Throughout, corresponding reference numerals are used for corresponding parts.

Further scope of applicability of the present invention will become apparent from the description given below. However, it should be understood that the detailed description and specific examples, while indicating preferred embodiments of the invention, are given by way of illustration only, since various changes and modifications within the spirit and scope of the invention will become apparent to those skilled in the art from this detailed description.

FIG. 1 shows an embodiment of a laser system 100 comprising a first laser resonator 101 and a second laser resonator 102. Both the first 101 and second 102 laser resonators are provided in the form of cavities. The first laser resonator 101 is bounded on one side by a first reflective element 114, such as a mirror. Analogously, the second laser resonator 102 is bounded by a second reflective element 115. The first laser resonator 101 and the second laser resonator 102 share a part of the cavity referred to as the common section 103. Within the common section 103, the first optical axis 116 and the second optical axis 117 are preferably substantially coinciding or parallel. The first laser resonator includes a first gain medium 104 of an Neodynium (Nd) doped host material such as Nd:YAG, capable of emitting light at a first wavelength of 1064 nm. The first gain medium 104 is located on the first optical axis 116. The second laser resonator 102 includes a second gain medium 105 on the second optical axis 117, comprising a Nd doped host material such as Nd:YAG, capable of emitting light at a second wavelength of 1319 nm. Suitable laser resonator configurations will be readily apparent to those skilled in the art given the benefits of this disclosure.

Other examples of suitable gain materials are Nd:YAP capable of lasing at about 1079 nm or at about 1341 nm, Nd:GdVO₄ capable of lasing at about 1064 nm or at about 1341 nm, or Yb:YAG emitting at about 1030 nm. Or alternatively crystal hosts such as YAG, forsterite, YAP, YVO4, LiCAF and KGW doped with active rare-earth or transition metal ions such as Nd, Er, Yb, Cr, and Ho. Optionally the host may be a silica glass, or fluoride glass.

The first gain medium 104 and the second gain medium 105 are not required to be of the same kind, such as the first gain medium 104 being of Nd:YAG emitting at 1064 nm and the second gain medium being of Nd:YAP 105 emitting at 1341 nm. Other suitable combinations will be suggested by the particular use to which the laser system 100 is being employed, and will be readily apparent to those skilled in the art given the benefit of this disclosure.

Inserted on the first and second optical axis 116, 117 in the common section 103 is an active common Q-Switch 106, which alters the quality factor (Q) of resonance for both the first wavelength and the second wavelength essentially simultaneously. A method of synchronizing two or more lasers simultaneously Q-Switched with a single active Q-Switch is described in WO 2008/006371 A1, which is hereby included as reference in its entity. The Q-Switch is an acousto-optical modulator controlled by an external RF-generator, or an electro-optical modulator controlled by a high-voltage generator. Alternatively, the common Q-switch 106 may be of passive type, comprising e.g. Cr:YAG, V:YAG and/or Cr:Forsterite.

The first optical field and the second optical field are combined to have substantially common optical axes. This may be accomplished by utilizing a beam combiner 121, such as a dichroic mirror which is essentially transparent for the first or the second wavelength while essentially reflecting for the other wavelength. For example, the mirror is essentially transparent for light with a wavelength of about 1064 nm, while reflectance for light with a wavelength of about 1319 nm is at least about 95%, such as at least about 99%. In this fashion the two beams may be overlaid. Another example of a beam combiner is a dispersing prism.

Essentially ending the common section, and thus both the first laser resonator 101 and the second laser resonator 102, is a common output coupler 107 that is partly reflecting to both the first optical field and the second optical field so as to couple out parts of the circulating laser fields. The common output coupler is partially reflective at the first wavelength of 1064 nm from the first laser resonator 101 and partially reflective for the second wavelength of 1319 nm emitted from the second laser resonator 102. For example in certain embodiments the reflectance for light with a wavelength of about 1064 nm is at least about 60%, such as at least about 65% or at least about 70%, or even at least about 75% or at least about 80%, while the reflectance at about 1319 nm is at least about 85%, such as at least about 90%, or even at least about 95%. Such an output coupler may be provided as a mirror that is readily created by known arts, and is for example accomplished by coating non-absorbing, transparent substrates with multiple layers of dielectric materials such as fluorides and oxides.

The common output coupler 107 is followed by a nonlinear medium 109 for sum frequency generation conversion of the first optical field and the second optical field into the third optical field. The nonlinear medium 109 for sum frequency generation is here implemented by an LBO crystal. The LBO may be arranged for non-critical phase matching. A typical length of the LBO crystal is in the range from about 10 mm to about 50 mm, such as in the range from about 15 mm to about 25 mm, or even about 20 mm.

The laser system 100 further comprises an output selector here implemented as a mirror selector 110 capable of holding a number of selection mirrors 111. The mirror selector 110 is capable of positioning a given selection mirror 111 to intersect the first optical axis 116, second optical axis 117 and third optical axis 118. The mirror selector 110 may comprise a wheel with slots around the circumference, into which the selection mirrors are mounted. Alternatively, it may comprise a linear stage with slots, into which the selection mirrors 111 are mounted. Or alternatively, it may comprise an axle with radially mounted arms or vanes appropriate for holding mirrors. The mirror selector 110 may comprise an electrical stepper motor, a linear motor, or an electrical motor. To assist exact positioning of the mirror selector 110 a disc with slots in combination with means for optically reading the disc position to establish the mirror selector position may e.g. be used. Optionally an electronic counter may be used for reading the mirror selector position. Other examples of mirror selectors 110 are readily apparent to those skilled in the art given the benefit of this disclosure.

The number of selection mirrors 111 in the mirror selector 110 is typically between 2 and 6, for example 4, optionally 3. The selection mirrors 111 are chosen as to enable control of which of the optical fields emitted from the laser system 100 are available at an output port 113. For instance, the optical fields transmitted through the selection mirrors 111 are routed to the output port 113, while the optical fields reflected are passed to a beam dump 112. Alternatively, the reflected optical fields are routed to the output port 113 of the laser system 100 while the transmitted optical fields are passed to the beam dump 112. In another alternative both the reflected and transmitted optical fields are used as output from the laser system 100, e.g. the reflected optical field being output from the output port 113, and the reflected optical field being output from a secondary output port.

In an example, where the transmitted fields are used as output from the laser system 100 a first selection mirror 111 may have a transmittance at a wavelength of about 589 nm of at least about 90%, such as at least about 95%, or even about 99.5%, and may have a reflectance at a wavelength of about 1064 nm of at least about 50%, such as at least about 75%, such as at least about 90%, or even at least about 99%. Finally, the first selection mirror 111 may have a reflectance at a wavelength of about 1319 nm of at least about 50%, such as at least about 75%, such as at least about 90%, or even at least about 99%.

A second selection mirror 111 may have a transmittance at a wavelength of about 589 nm of at least about 90%, such as at least about 95%, or even about 99.5%, and may have a reflectance at a wavelength of about 1064 nm of at least about 50%, such as at least about 75%, such as at least about 90%, or even at least about 99%, and finally may have a transmittance at a wavelength of about 1319 nm of at least about 50%, such as at least about 75%, such as at least about 90%, or even at least about 99%.

A third selection mirror 111 may have a transmittance at a wavelength of about 589 nm of at least about 90%, such as at least about 95%, or even about 99.5%, may have a transmittance at a wavelength of about 1064 nm of at least about 50%, such as at least about 75%, such as at least about 90%, or even at least about 99%, and further may have a reflectance at a wavelength of about 1319 nm of at least about 50%, such as at least about 75%, such as at least about 90%, or even at least about 99%.

A fourth selection mirror position may be empty so as to allow all wavelengths to pass through. Optionally the selection mirror position may hold a mirror with a reflectance at a wavelength of about 589 nm and at a wavelength of about 1064 nm and at a wavelength of about 1319 nm, all at least about 95% or at least about 99%.

The laser system 100 may optionally comprise an exposure control device 108 to control the time the emitted wavelengths are accessible at the output port 113. This may be utilized to deliver a pulse of energy sufficient for the intended treatment. The exposure control device 108 may comprise a mechanical shutter to control the duration of the light emission. Alternatively, the exposure control device 108 may be an acousto-optical device capable of deflecting the emitted beam onto a beam dump. The exposure control device 108 may also be an electro-optical device capable of altering a polarization state of the beam in combination with a device capable of passing a given polarization state onto the output port of the system, while being capable of deflecting the orthogonal polarization state onto a beam dump. Other types of exposure control devices 108 to control the length of the pulse train delivered onto the output port 113 of the laser system 100 will be readily apparent to those skilled in the art given the benefits of this disclosure.

FIG. 1 indicates various positions for the exposure control device 108; either between the output coupler and the nonlinear medium; between the nonlinear medium and the output selector, here shown as a mirror selector 110; or between the mirror selector 110 and the output port 113 of the laser system 100.

This embodiment of the laser system 100 enables emission of any combination of the three wavelengths 589 nm, 1064 nm and 1319 nm, simultaneously, as well as any of the wavelengths individually.

The one or more optical pump sources may, for instance, be fiber-coupled laser diodes. Typical pump wavelengths may be about 808 nm or about 885 nm. Further information about a suitable pump configuration may be found in WO 2008/006371 A1, which is hereby incorporated by reference.

FIG. 2 shows another embodiment of a laser system 200 according to the invention, corresponding to the embodiment shown in FIG. 1. Therefore only the differences between the two embodiments are discussed here. As in the embodiment shown in FIG. 1, the laser system 200 comprises a first laser resonator 201 and a second laser resonator 202. However, in this embodiment, the two laser resonators 201, 202 do not have a common section, but are separate. The first laser resonator 201 comprises a first gain medium 204, a first reflective element 214, and a first output coupler 2071. Correspondingly, the second laser resonator 202 comprises a second gain medium 205, a second reflective element 215, and a second output coupler 2072. The first laser resonator 201 and the second laser resonator 202 are here shown to comprise a first Q-switch 2061 and a second Q-switch 2062, respectively. The Q-switches 2061, 2062 may be of an active type as discussed for the embodiment in FIG. 1.

The first optical field and the second optical field output from the first and second laser resonators are combined with a beam combiner 221 and launched into the nonlinear medium 209 as in the above embodiment. Within the nonlinear medium 209, the third optical field is generated by sum frequency generation from the first optical field and the second optical field. For example, 589 nm light generated from 1064 nm and 1319 nm light.

The laser system 200 may furthermore comprise an exposure control device 208 and an output selector 210, as in the above embodiment.

FIG. 3 schematically illustrates an embodiment of a laser system 300 according to the invention, wherein the laser system 300 comprises a hand piece 330 connected to the output port 313 for delivering the first optical field, the second optical field, and/or the third optical field to a target skin area on the skin 332 of a subject. The hand piece 330 is connected to the output port by a beam delivery component 331, such as an optical fiber, a fiber bundle, or a waveguide. The hand piece 330 may optionally contain one or more optical components 333, such as lenses and/or mirrors, e.g. for focusing the laser light from the laser system 300 onto the skin 332 so as to stimulate the desired reaction in the skin. For example, the hand piece may focus the laser light onto a spot of about 1 mm diameter to stimulate collagen growth beneath the epidermis, or to cause thermal damage to vessels in the dermis to cause necrosis of the vessel, without damaging surrounding tissue. In certain embodiments the focus spot diameter can be altered by the operator to focus the laser light to spot sizes in the range of from about 0.5 mm to about 5 mm in diameter, such as from about 0.6 mm to about 3 mm in diameter, for example about 0.6 mm, about 0.8 mm, about 1.2 mm, about 1.6 mm, about 2.0 mm, or even about 2.5 mm.

As illustrated in FIG. 4, the hand piece 330 may furthermore comprise an active scanning device for scanning the laser light over a preset treatment area of the skin 332 in a preset pattern 335 of target skin areas (“spots”). For example, the laser light may be scanned in a dotted non-overlapping continuous pattern over an area of about 1 cm by about 1 cm to cause a fractional effect for collagen stimulation. In another example, the laser light may be continuously swept in lines across an area of about 0.5 cm by 0.5 cm to cause vessel necrosis. In yet another example the laser light may be scanned in a random non-overlapping dot pattern over an area of about 1 cm by about 1 cm to cause a fractional effect for collagen stimulation without causing excessive heating to the surrounding tissue. Other examples of scanning patterns are readily apparent to those skilled in the art given the benefits of this disclosure.

As an example, for inducing the necessary micro-thermal damages stimulating the healing response in the skin it is preferable to deliver the light energy in distinct non-overlapping spots on the skin, whereby the beam is rapidly moved between these target skin areas. The beam is held in the position of each individual spot for a preset time duration (also called dwell time) to allow the sufficient energy to be delivered to the skin to cause the desired micro-thermal damage. Thus for treating larger areas of the skin, it is preferable to use an automated hand piece 330, comprising means for scanning and focusing the beam 333 onto the skin 332 in a pre-programmed pattern 335 of individual spots 334. The hand piece 330 may be optically connected to the laser system 300 by means of e.g. a fiber 331. The selected treatment area should be of a size allowing efficient treatment of larger skin areas by subdivision, yet at the same time not larger than the duration of the scan over the area may be well tolerated by the patient. For example a treatment area of about 10 mm-by-10 mm with a spot pattern of for example 5-by-5 spots, or any other desirable number, organized in a rectangular fashion 335. Preferentially, the individual spots or target skin areas are not accessed in a direct sequential order, but in such a way as to ensure neighboring spots not being treated in direct sequence. This approach is known to reduce risk of collateral thermal damage. Spots 334 may be focused to a spot diameter in the range from about 0.05 mm to about 5.0 mm; this range is known to be of significant relevance in inducing the micro-thermal damages in the skin.

The main absorbing constituents of the skin, here referred to as the chromophores of the skin, are hemoglobin, melanin, lipids and fat, and water. Often it is desirable to select a wavelength which selectively target one chromophore but may be able to pass preferably unhindered through the non-targeted chromophores. Absorption curves for the main chromophores of the skin are shown in FIG. 5. Absolute absorption coefficient are shown for A hemoglobin in oxygenated (dashed line) and deoxygenated (solid line) forms; B different constituents of melanin; C typical skin lipids/fats; and D water at 25 C. As different wavelengths have different penetration depths in the skin and target different chromophores, treatments may beneficially utilize two or more of the wavelengths available from the laser system disclosed herein delivered in essence simultaneously.

FIG. 6 illustrates the dermis 440 and epidermis layers 441 in skin 432. The epidermis is in itself constituted by a number of individual layers—the most important in the respect of light based treatments being the layers responsible for the pigmentation of the skin, namely the basal cell layer 444 in which melanin production takes place, and the spinosum layer 445 which is the main melanin containing layer. Upper layers of the dermis 446 are responsible for mechanical wear resistance and contain in general very low levels of water. The epidermis does not contain any blood vessels or fluid supply for the skin; neither does it contain the collagen matrix that gives the skin its elasticity and tear resistance. The dermis 440 forms the basis of the skin and contains the collagen threads 443 giving elasticity to the skin, as well as the vessels 442 supplying the skin with fluids and nutrition. The dermis furthermore contains the sebaceous glands 458 and the hair follicles 456 through which the sebaceous glands are connected to the skin surface 432. Furthermore, the penetration depths of the representative wavelengths (589 nm, 1064 nm, 1178 nm, and 1319 nm), corresponding to the first optical field, the second optical field, and the third optical field, are indicated along with blood vessels 442, collagen fibers 443 and a sebaceous gland 458.

It is desirable to select wavelengths for skin rejuvenation according to the absorbance graphs shown in FIG. 5. For example, for efficiently stimulating the collagen regrowth by stimulating the healing response by inducing localized micro-thermal injuries in the skin, it is desirable to have the light energy evenly absorbed through the depth of the skin. The invention features, in one aspect, a safe and effective method and apparatus for skin rejuvenation by utilizing a select mixture of wavelengths and energies in accordance with the absorbance graphs in FIG. 5. For example, referring to FIG. 5, FIG. 6a and FIG. 6B, wavelengths in the visible range between 570 nm and 600 nm will preferentially be absorbed in the hemoglobin 442 and may thus be used for efficiently heating of these chromophores. Longer wavelengths may be used to preferentially heating other chromophores in the skin. For example, wavelengths in the 1000 nm to 1100 nm range have very low absorption in the different constituents of the skin and may thus be utilized to give an in-depth heating of the dermis 440. Wavelengths in the range 1100 nm to 1200 nm have high absorption in skin fats and lipids and may thus be used for preferentially heating fat containing regions of the skin, e.g. sebaceous glands 458. This may be used for reducing occurrence of acne vulgaris. Wavelengths in the range 1300 nm to 1400 nm have higher absorption in water than wavelengths in the 1000 nm to 1100 nm range and may thus be used for preferentially heating the upper layers of the dermis 440. Wavelengths longer than 1400 nm have significantly higher absorption in water and are thus not preferable for heating in the depth of the skin as penetration depth is negligible.

Also, a beam combining one or more of the above wavelengths may have synergistic effects. For example, for treatments, wherein hemoglobin present in deeper layers of the dermis should preferentially be targeted, such as in deeper lying broken vessels, a first laser beam may modify the optical properties of a second laser beam, for example by inducing clots or met-hemoglobin, which in essence works to increase the absorption of a second laser beam. For example light in the wavelength range of 500 nm to 600 nm may produce clots and met-hemoglobin in hemoglobin, while light in the wavelength range of 1030 nm to 1080 nm will experience a significant increase in absorption in hemoglobin following the production of clots and met-hemoglobin.

Thus by combining three or more appropriately selected wavelengths in a single beam the individual and synergistic combined beneficial properties of the different laser wavelengths may be exploited in a single treatment. For example, a skin rejuvenation treatment may be performed with a single pass only. A single pass treatment may include simultaneously delivering to the target skin area a first beam of radiation to treat vascular abnormalities in combination, a second beam of radiation to induce micro-thermal damages in the lower layers of the dermis and a third beam of radiation to induce micro-thermal damages in the upper layers of the dermis or a third beam to introduce micro-thermal damages in fat containing tissues such as sebaceous glands 458. For example, referring to FIG. 6a and FIG. 6b, a treatment may comprise delivering to the target skin area a beam of light comprising 589 nm and 1064 nm and 1319 nm light (a) for delivering energy specifically to hemoglobin 442, the lower parts of the dermis 440, and the upper parts of the dermis 440, respectively; or comprising 589 nm, 1064 nm and 1178 nm light (b), for delivering energy specifically to hemoglobin 442, the lower parts of the dermis 440 and to fat containing tissues 458, respectively. This significantly decreases treatment time and patient discomfort while additionally improving tissue response by simultaneously targeting multiple chromophores and absorbers and thus inducing heating evenly distributed throughout the thickness of the dermis.

Laser systems 100, 200, 300 in accordance with those described herein may be utilized to treat a range of skin conditions. Conditions include as examples conditions such as, but not limited to, wrinkled skin, aged skin, sun damaged skin, pigmented lesions, acne vulgaris, vascular lesions and other blood vessel related conditions such as, but not limited to, hemangioma, telangiectasis, and striae (reddish stretch marks).

FIG. 7a-7f illustrates all wavelength combinations possible with the inventive system. Indicated in the figures are individual peaks 550 of the optical fields and the pulse envelope 551 of a pulse train. Output pulses, e.g. as defined by the exposure control device, are either emitted as a train of peaks being referred to as a pulse train having a pulse envelope 551, or as long single pulses. If a third optical field is to be generated to achieve the desired output, corresponding to the case in FIG. 7a-7d, the first optical fields and second optical fields are emitted as trains of peaks, in order to achieve sufficiently high peak powers to achieve the desired nonlinear conversion. However, if the third optical field is not desired at the output, corresponding to the case in FIG. 7e-7g, the first optical field and second optical field may be emitted as a continuous wave (CW) signals, as indicated in the figures. Throughout this document, when discussing light at different wavelengths being delivered simultaneously, this refers to temporally overlapping pulse envelopes 551, but not necessarily to overlapping pulse train peaks 550.

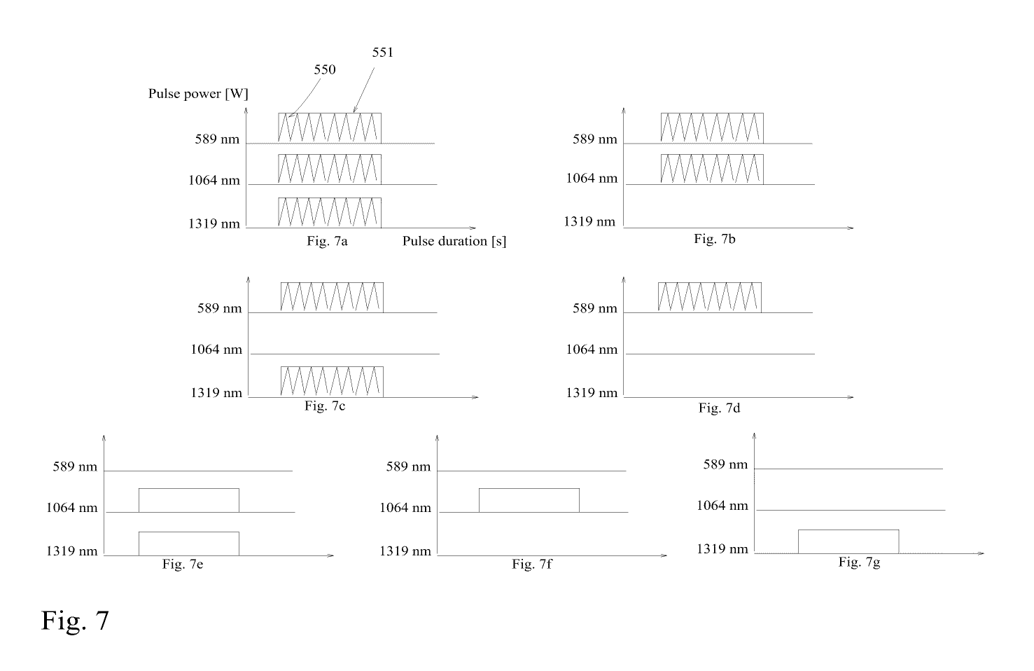

As an example the treatment of wrinkles is exemplified in FIG. 8a. The laser system is set to deliver laser light as a pulse train consisting of light having wavelengths of about 1319 nm, about 1064 nm, and about 589 nm simultaneously, with a pulse train duration of about 5 to about 200 milliseconds, such as about 20 milliseconds. The laser light is delivered to the hand piece by the optical fiber. By means of lenses the hand piece focuses the laser light onto a spot with a typical size of about 1 mm diameter or less. The light delivered may have a fluence at the three wavelengths of about 8 J/cm² or less at a wavelength of about 589 nm, about 12 J/cm² or less at a wavelength of about 1319 nm, and about 12 J/cm² or less at a wavelength of about 1064 nm. As the individual wavelengths have different absorption profiles in the skin the targeted chromophores and penetration depths are different for each individual wavelength. Light at a wavelength of about 589 nm is preferentially absorbed in hemoglobin and may cause a heating in the upper dermis, light at a wavelength of about 1064 nm has low absorption in both fatty tissue, water and hemoglobin and thus may penetrate deep into the dermis, while light at a wavelength of about 1319 nm has higher water and lipid absorption than light at a wavelength of about 1064 nm and thus is absorbed at higher layers in the dermis. The result causes a heating effect and/or other effects throughout the dermis triggering collagen regrowth throughout the dermis.

Another example hereof is treatment of reddish acne scarring. Here the treatment utilizing the disclosed laser system benefits from being able to close the superficial vessels and thus removing the reddishness with laser light having a wavelength of about 589 nm, and simultaneously stimulate new collagen growth for filling the scar by irradiation throughout the depth of the dermis by the method described above. Utilizing multiple simultaneous wavelengths thus may provide a faster, more efficient method for treatment of skin conditions.

Furthermore treatments likely benefit from using multiple simultaneous wavelengths through changes in the optical properties of the skin of one wavelength mediated by another wavelength.

Without wishing to be bound by theory it is believed that essential simultaneous delivery of multiple wavelengths may enhance or improve the treatment efficiency beyond what may be achieved by solely delivering the same wavelengths individually and sequentially.

As an example hereof is treatment of vessels. Deep lying and or larger vessels are traditionally treated with pulses having a wavelength of 1064 nm due to the otherwise more efficient pulses in the wavelength range of 570-600 nm not having necessary depth penetration in the skin. This may have significant side effects, such as purpura and pain, from having low hemoglobin absorption of 1064 nm and thus needing very high fluences. However blood in the form of Oxy-hemoglobin may be converted to Met-hemoglobin by applying light with a wavelength of about 589 nm causing an initial heating of the blood cells. This causes a transition from Oxy-hemoglobin to Met-hemoglobin which takes place in essence instantaneously. Met-hemoglobin has a significantly higher absorption of light with a wavelength of 1064 nm than Oxy-hemoglobin, which may be utilized to use simultaneously delivered 1064 nm light to cause vessel necrosis. Overall the treatment may likely benefit from lower side effect profile due to more controlled and selective energy delivery to the target vessel.

The invention is defined by the features of the independent claim(s). Preferred embodiments are defined in the dependent claims. Any reference numerals in the claims are intended to be non-limiting for their scope.

Some preferred embodiments have been shown in the foregoing, but it should be stressed that the invention is not limited to these, but may be embodied in other ways within the subject-matter defined in the following claims. For example, the system have been discussed primarily in embodiments suitable for generating light at, e.g. about 1064 nm, about 1319 nm, and about 589 nm. However, many other output wavelengths may be generated with the inventive system by a proper choice of gain media and nonlinear media.

1. A laser system for skin treatment, the laser system comprising:
a first laser resonator comprising at least a first gain medium for generating a first optical field along a first optical axis, the first laser resonator further comprising a first reflective element and a partly reflective first output coupler,
a second laser resonator comprising at least a second gain medium for generating a second optical field along a second optical axis, the second laser resonator further comprising a second reflective element and a partly reflective second output coupler,
at least one nonlinear medium for generating a third optical field along a third optical axis via a nonlinear interaction between the first optical field and the second optical field,
at least one optical output port capable of delivering one or more of said optical fields to an output, and
at least one optical pump source for optically pumping the first gain medium and the second gain medium, wherein the first laser resonator comprises at least one first Q-switch and/or wherein the second laser resonator comprises at least one second Q-switch, the first Q-switch and/or the second Q-switch being capable of controlling a resonance quality of the first laser resonator and/or second laser resonator, respectively, wherein the first laser resonator and the second laser resonator are arranged to have substantially overlapping optical axis in a common section over at least a part of a length of the first laser resonator and a part of a length of the second laser resonator, and wherein the first output coupler and the second output coupler are provided as a common output coupler, and wherein the nonlinear medium comprises at least one nonlinear crystal.

2. The laser system according to claim 1, wherein the first Q-switch and/or the second Q-switch is/are of an active type.

3. The laser system according to claim 1, wherein the first Q-switch and/or the second Q-switch are of a passive type.

4. The laser system according to claim 1, wherein the first laser resonator comprises at least one additional first gain medium and/or wherein the second laser resonator comprises at least one additional second gain medium.

5. The laser system according to claim 1, wherein at least one of the first gain medium and/or the second gain medium comprises a rare-earth doped crystal.

6. The laser system according to claim 1, wherein the first laser resonator is adapted for lasing with a first wavelength in the range from about 1020 nm to about 1080 nm, and wherein the second laser resonator is adapted for lasing with a second wavelength in the range from about 1300 nm to about 1350 nm.

7. The laser system according to claim 1, the laser system further comprising at least one output selector for selecting the first optical field, the second optical field, and/or the third optical field to be delivered to the output port.

8. The laser system according to claim 7, wherein the output selector comprises a mirror selector and a plurality of selection mirrors, the mirror selector being capable of selectively positioning any of the selection mirrors so that the incident first optical field, second optical field and third optical field are divided in to reflected fields and transmitted fields, and wherein at least one of the reflected fields or the transmitted fields is/are delivered to the output port.

9. The laser system according to claim 1, wherein the first Q-switch and the second Q-switch are comprised as a common Q-switch.

10. The laser system according to claim 1, wherein the nonlinear medium is capable of generating the third optical field by sum frequency generation from the first optical field and the second optical field.

11. The laser system according to claim 1, wherein the first gain medium is pumped substantially along the first optical axis and/or the second gain medium is pumped substantially along the second optical axis.

12. The laser system according to claim 1, wherein the first gain medium and/or the second gain medium is side-pumped.

13. The laser system according to claim 1, wherein the pump source comprises one or more laser diodes.

14. The laser system according to claim 1, wherein the laser system further comprises a hand piece, the hand piece being optically connected to the output via a beam delivery component.

15. The laser system according to claim 14, wherein the beam delivery component comprises an optical fiber, and wherein the fiber has a core diameter in the range from about 50 μm to about 400 μm.

16. The laser system according to claim 14, wherein the hand piece is adapted to deliver laser light from the output onto a treatment area of skin,
the hand piece being configured for scanning the laser light in a pre-set pattern of individual target skin areas covering said treatment area.

17. A method for treatment of skin, the method comprising:
selecting a target skin area to be treated,
positioning a beam delivery device in proximity to the target skin area,
irradiating the target skin area with laser light having at least three distinct wavelengths simultaneously or in succession.

18. The method according to claim 17, wherein the target skin area is irradiated by at least one pulse of the laser light, the laser light having at least three distinct wavelength components suitable for heating constituents in the skin.

19. The method according to claim 17, wherein the treatment is cosmetic.

20. The method according to claim 17, wherein the treatment is photo rejuvenation of skin.

21. The method according to claim 17, wherein the distinct wavelengths are chosen to be a first wavelength in the range from about 570 nm to about 610 nm, a second wavelength in the range from about 1020 nm to about 1100 nm, and a third wavelength in the range from about 1140 nm to about 1220 nm or from about 1300 nm to about 1400 nm, respectively.

22. The method according to claim 17, wherein the laser light is provided by a laser system according to claim 1.

23. The method according to claim 17 further comprising irradiating the target skin area for a duration in a range from about 5 ms to about 300 ms.

24. The method according to claim 17 wherein said laser light delivers to the target skin area a total radiant exposure in a range from about 15 J/cm² to about 150 J/cm².

25. The method according to claim 21 wherein said laser light delivers to the target skin area a radiant exposure in a range of about 5-50 J/cm² for said first wavelength, of about 5-50 J/cm² for said second wavelength, and of about 5-50 J/cm² for said third wavelength.

26. The method according to claim 17 wherein the light at least at one of the distinct wavelengths comprises a pulsed laser beam forming a pulse train having a pulse repetition frequency, and wherein the pulse repetition frequency is in the range from about 8 kHz to about 75 kHz.

27. The method according to claim 17 wherein the laser light is delivered to a treatment area comprising multiple target skin areas by a hand piece receiving a beam from a light source such as to deliver the beam onto the treatment area, the method further comprising
the hand piece scanning the beam in a pre-set pattern of individual target skin areas covering the treatment area.

28. The method according to claim 27 wherein said individual target skin areas of said pre-set scanning pattern are addressed in a non-sequential order and wherein said non-sequential order includes not irradiating neighboring target skin area positions in direct sequence.

29. The method according to claim 27 wherein a dwell time at said individual target skin area positions are in the range from about 5 ms to about 100 ms.

30. The method according to claim 17 wherein the size of said target skin area on the skin is in the range from about 0.8 mm to about 5.0 mm.

31. The method according to claim 27 wherein the size of said individual target skin areas on the skin is in the range from about 0.05 mm to about 0.4 mm.
